(19) United States
(12) Patent Application Publication
(10) Pub. No.: US 2002/0192205 A1
(43) Pub. Date: Dec. 19, 2002

(54) Immunogenic compositions of low molecular weight hyaluronic acid and methods to prevent, treat and diagnose infections and diseases caused by group A and group C streptococci

(76) Inventors: Francis Michon, Bethesda, MD (US); Samuel Moore, Sykesville, MD (US); Maryline Laude-Sharp, Olney, MD (US); Milan Blake, Fulton, MD (US)

Correspondence Address: MORGAN & FINNEGAN, L.L.P., 345 Park Avenue, New York, NY, 10154-0053, US

(21) Appl. No.: 09/853,367

(22) Filed: May 11, 2001

(51) Int. Cl. A61K 38/46; A61K 39/095

(52) U.S. Cl. 424/94.62

(57) ABSTRACT

The present invention provides antigenic compositions and methods for treatment and prevention of infection and disease caused by group A and group C streptococci. In particular, the invention provides low molecular weight hyaluronic acid, low molecular weight hyaluronic acid linked to a carrier and compositions comprising them. The compositions elicit antibodies to low molecular weight hyaluronic acid which are cross-reactive with group A and C streptococci and which are minimally cross-reactive with native hyaluronic acid. The invention is particularly useful for providing both active and passive immunogenic protection for those infected with or at risk of infection with group A and group C streptococci. Additionally, the present invention provides methods and compositions useful for diagnosing infections and diseases caused by group A and group C streptococci.

### FIELD OF THE INVENTION

This invention relates to hyaluronic acid/polypeptide conjugate molecules, and pharmaceutical compositions comprising them. In particular the present invention relates to hyaluronic acid, and more preferably, low molecular weight hyaluronic acid (LMW-HA), and LMW-HA/polypeptide conjugate molecules that elicit antibodies to hyaluronic acid which are cross-reactive with both group A and group C streptococci. The molecules of the invention and pharmaceutical compositions comprising them are useful for the treatment and prevention of infection and for the diagnoses of disease caused by group A and group C streptococci.

### BACKGROUND OF THE INVENTION

Hyaluronic acid (HA) is a naturally occurring glycosaminoglycan. It is made up of repeating units of N-acetylglucosamine and glucuronic acid. See FIG. 1. HA occurs in animal tissue, e.g. spinal fluid, ocular fluid, synovial fluid, skin, and also in some streptococci, such as in the capsules of group A and group C streptococci. Such mucoid or highly encapsulated strains of group A streptococci have been associated both with unusually severe infections, and with acute rheumatic fever (Johnson et al, 1992, J. Infect. Dis. 166:374-382). Human invasive soft-issue infections caused by group A and group C streptococci are associated with significant morbidity and mortality. Group C streptococci are also associated with pharyngitis and reactive arthritis. Further, group C streptococcal infections are prevalent in horses.

The mucoid colony morphology of group A and group C streptococci is a result of abundant production of capsular polysaccharide composed of hyaluronic acid. The hyaluronic acid capsule of group A streptococci has recently been shown to exhibit a number of important roles in the pathogenicity of these organisms. Among other things, the HA capsule protects mucoid group A streptococci from phagocytosis and has an important role in virulence (Wessels et al. 1991, Proc. Natl. Acad. Sci. USA 88: 8317-21; Dale et al. 1996, Infect. Immunol. 64:1495-501; and Moses et al. 1997 Infect. Immumol 65;64-71). In addition, the HA capsule modulates M protein-mediated adherence and acts as a ligand for the attachment of group A streptococcus to CD44 on human keratinocytes. The group A streptococcal HA capsule is both highly conserved and surface-exposed which indicates that HA may serve as a universal adhesion site for the attachment of other strains of bacteria to the pharyngeal mucosa and to the skin (Schrager et al 1998, J. Clin. Invest. 101:1708-16).

Preventing and treating infections of gram-positive pathogens such as streptococci are particularly important because of the development of resistant strains which are both difficult to treat and difficult to eradicate once established. Although conjugation of polysaccharide antigens, or of immunologically inert carbohydrate haptens, to thymus dependent (TD) antigens such as proteins enhances their immunogenicity, it was not evident whether such an immunogenic response, if elicited against HA, would provide protection against HA containing bacteria, such as group A or group C streptococci. Further, the presence of HA both in mammalian tissue and on streptococci complicates the development of HA as a carbohydrate antigen to treat or prevent streptococcal infection because of a potential to elicit an autoimmune response directed at the host tissue.

Until recently, HA has been thought to be a nonimmunogenic molecule (Meyer, 1936, J. Biol. Chem. 114:689 and Humphreys, 1943, Biochem J. 37:460). However, more recent studies indicate that naturally occurring antibodies to HA are present in several species (Underhill, 1982, Biophys. Res. Commun. 108:1488). Fillit et al. induced antibodies to HA in mice with HA bound to liposomes. They report that HA is immunogenic and identified two antigenic determinants on the molecule. Fillit et al. also noted that the mode of presentation of HA on the liposome is important to its immunogenicity. Finally, Fillit et al. reported that an HA antibody was cross-reactive with heparan sulfate and that such cross-reactions could be involved in the pathogenesis of autoimmune vascular disease (Fillit et al., 1988, J. Exp. Med. 168:971-982). Based on the reports summarized above, it remains unpredictable whether HA or HA conjugates would elicit an immune response useful for inhibiting or treating infection from HA containing bacteria such as group A or group C streptococci.

### SUMMARY OF THE INVENTION

The present invention provides for an immunogenic composition comprising HA and LMW-HA conjugated to a polypeptide or protein carrier. The conjugate molecules of the present invention are useful for eliciting antibodies that are cross-reactive to bacteria containing HA, such as both group A and group C streptococci. The conjugate molecules of the present invention and pharmaceutical compositions comprising them are useful in a method for the treatment and diagnoses of infection and disease caused by such bacteria, including group A and group C streptococci.

Applicants have surprisingly discovered that the LMW-HA conjugates are immunogenic in mammals. Even more surprisingly, applicants discovered that antibodies elicited by the conjugates of the invention are cross-reactive with groups A and C streptococci but are only minimally cross-reactive with native HA associated with mammalian tissue.

Methods for conjugating LMW-HA to polypeptides include reductive amination, treatment with cyanogen bromide, amide bond formation between a free amino group on the carrier and a carboxylate moiety on the LMW-HA, or by use of a linker molecule. The invention provides pharmaceutical compositions comprising conjugate molecules of LMW-HA and the use of these compositions to elicit antibodies for the treatment of infection by HA containing bacteria such as groups A and C streptococci, as vaccines, and for diagnostics. Any polypeptide that converts a carbohydrate T cell independent response to a T cell dependent response is suitable for use as a carrier. Examples are toxins or toxoids such as tetanus toxoid, diphteria toxoid, and pertussis toxins or toxoids, neisserial porins, e.g., PorA of gonococci, and PorB of meningococci.

The present invention also provides pharmaceutical compositions, vaccines and other immunological reagents derived from the immunogenic LMW-HA-polypeptide conjugates.

The invention is further directed to a method of immunizing a mammal against bacterial infections. The method comprises administrating an effective amount of the pharmaceutical composition of the invention to a mammal for deterring infection from a disease causing organism. The methods are useful for preventing or treating infection by bacteria containing HA, such as group A and group C streptococci.

The invention also provides a method of eliciting antibodies in mammals, preferably humans, with the inventive LMW-HA-polypeptide conjugates. The invention also provides for an immunoglobulin composition and isolated antibody that are elicited in response to immunizing a mammal using the polysaccharide-polypeptide conjugates of the invention. Such immunoglobulin and isolated antibody are useful as therapeutic agents and as diagnostic reagents.

The immunoglobulins and antibodies produced are specific for LMW-HA. The conjugate molecules of the invention are also useful for raising monoclonal and antiidiotypic antibodies according to well known methods.

### BRIEF DESCRIPTION OF THE DRAWINGS

FIG. 1: Structure of hyaluronic acid (HA).

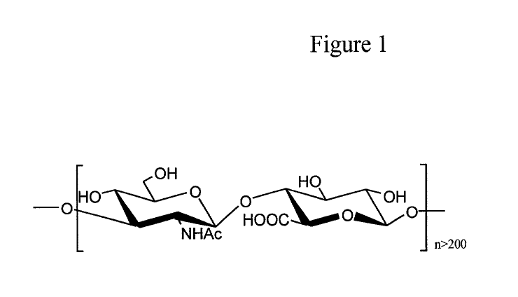

FIG. 2: Generation of antigenic low molecular weight LMW-HA by sonication and/or acid treatment.

FIG. 3: Structure of the streptococcal HA protective epitope; a) tetrasaccharide from sonicated HA; b) tetrasaccharide generated after the action of hyaluronidase.

FIG. 4: ELISA titers of rabbit antisera to sonicated HA-TT conjugates.

FIG. 5: ELISA titers of rabbit antisera to acid hydrolyzed LMW-HA/TT conjugates.

FIG. 6: Titration of rabbit antisera to sonicated LMW-HA/TT conjugate.

FIG. 7: Titration of rabbit antisera to acid hydrolyzed LMW-HA/TT conjugate.

FIG. 8: Inhibition of rabbit antiserum # 75295 with acid hydrolyzed HA, sonicated HA, and sonicated HA conjugate on LMW-HA/HSA coated plates.

FIG. 9: Inhibition of rabbit antiserum # 75295 with sonicated HA, native HA, D-glucuronic acid, HA disaccharide, and HA tetrasaccharide on LMW-HA/HSA coated plates.

FIG. 10: Inhibition of rabbit antiserum # 72700 with sonicated HA, native HA, D-glucuronic acid, HA disaccharide, and HA tetrasaccharide on LMW-HA/HSA coated plates.

FIG. 11: Inhibition of rabbit antiserum # 72700 with sonicated HA, native HA, HA disaccharide, HA tetrasaccharide, and HA hexa/octasaccharide on LMW-HA/HSA coated plates.

FIG. 12: ELISA titers of BALB/c mouse antisera to LMW-HA/rPorB conjugate.

FIG. 13: ELISA titers of CD1 mouse antisera to LMW-HA/rPorB conjugate.

FIG. 14: Passive immunization of Balb/c mice with rabbit antisera; challenge with GAS type 6 (4,400 cfu/mL). Respectively, ▪ rabbit antisera against PBS/CFA; ♦ rabbit antisera against sonicated LMW-HA/TT; ● rabbit antisera against sonicated LMW-HA/TT; ▴ rabbit antisera against sonicated LMW-HA/TT.

FIG. 15: Passive immunization of Balb/c mice with rabbit antisera; challenge with GAS type 3 (2.5×10⁵ cfu/mL). Respectively, ▪ rabbit antisera against PBS/CFA; ♦ rabbit antisera against sonicated LMW-HA/TT; ● rabbit antisera against sonicated LMW-HA/TT; ▴ rabbit antisera against sonicated LMW-HA/TT.

### DETAILED DESCRIPTION OF THE INVENTION

Hyaluronic acid is used as an immunogen to raise a protective and/or therapeutic response. In particular, HA is useful for raising an immune response that is cross-reactive with bacteria, such as group A and group C streptococci, that have HA on their surface. Without being bound by theory, it is believed that the epitope cross-reactive with group A and group C streptococci is about 3 or 4 residues in length and is located at the nonreducing terminal. There does not appear to be a significant difference whether the non-reducing terminal glucuronic acid residue is saturated or unsaturated as both epitopes are protective. In addition, it appears that the terminal glucuronic acid is converted to unsaturated glucuronic acid in blood and other body fluids. HA can terminate in either a glucosaminyl or glucuronyl residue, the immune response is enhanced when the percentage of glucuronic acid or unsaturated glucuronic acid at the nonreducing terminal of HA is increased over the percentage of N-acetylglucosamine.

Although native HA may be used for preparing conjugates according to this invention, preferably LMW-HA that is from about 3 or 4 saccharides to about 2000 saccharides or from about 600 daltons to about 400 Kd in size is used to prepare conjugates. More preferably, the LMW-HA is about 4 saccharides or 2 repeat units to about 100 repeat units or about 800 daltons to about 40 Kd in size. A most preferred size for the LMW-HA is about 4 repeat units to about 10 or about 20 repeat units or about 800 daltons to about 4 or 8 Kd. The LMW-HA can be obtained from native HA, which typically has a molecular weight of 400 Kd to several million daltons (available from Sigma or by purification according to U.S. Pat. No. 4,141,973), by various methods including sonication (Kubo K; et al. Glycoconj J., 1993, 10(6):435) or by chemical (Blatter G, Carbohydr. Res. 1996, 288:109-125 and Halkes K. M. Carbohydr. Res. 1998, 309: 161-164) and/or enzymatic (De Luca et al. J. Am. Chem. Soc. 1995 117:5869-5870) methods.

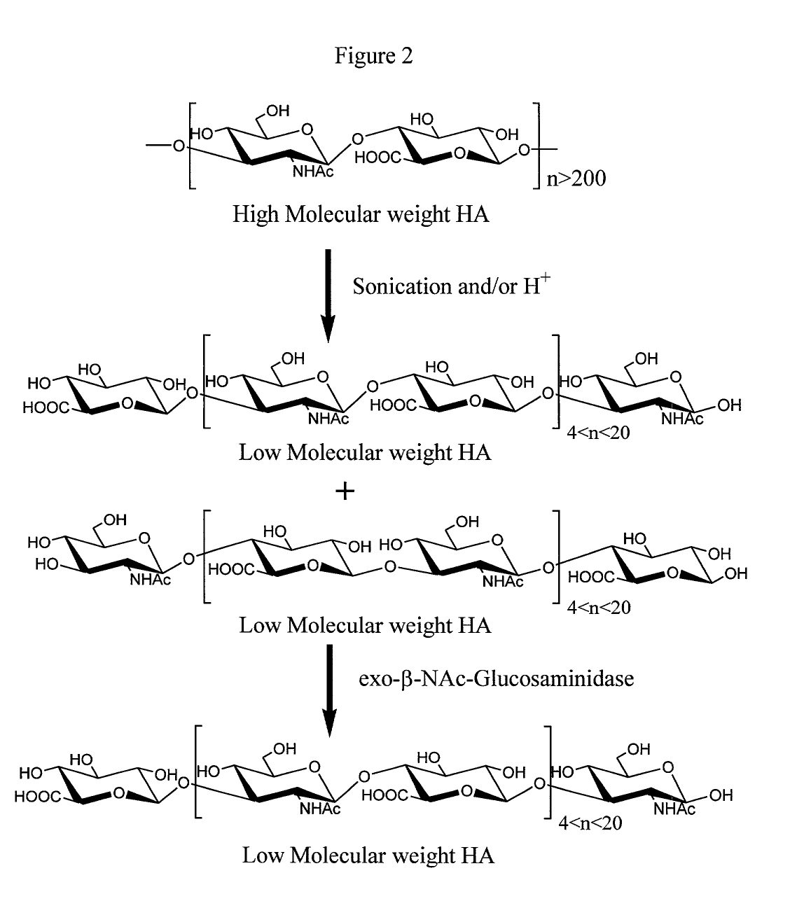

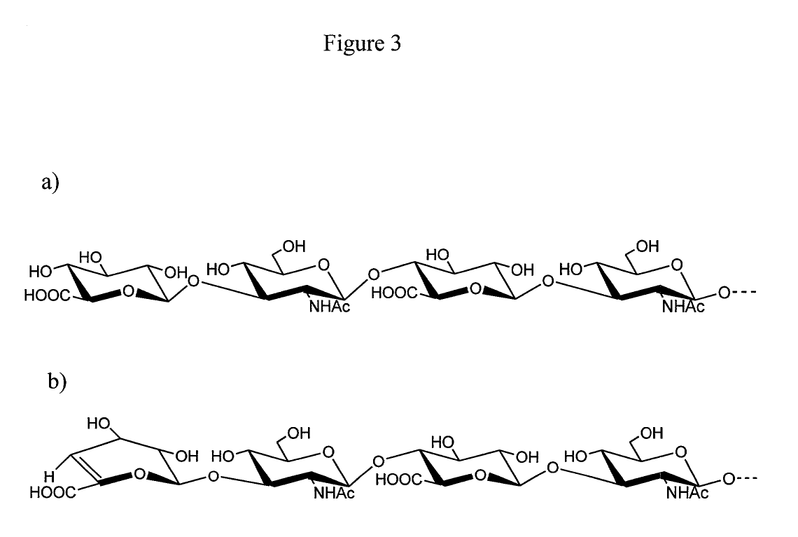

The invention also provides for LMW-HA molecules containing glucuronic acid residues at the nonreducing terminal. One method for obtaining glucuronic acid terminal HA fragments is by sonication of native HA according to the method of Kubo K; et al. Glycoconj J., 1993, 10(6):435. The percentage of molecules with a glucuronic acid terminal can be increased by treatment of the sonication product with an exo-β-N-acetyl glucosamimidase to remove any non-reducing terminal N-acetyl glucosaminyl residues and provide LMW-HA with a higher percentage of glucuronyl residues at the nonreducing terminal of the molecules. An alternative method for obtaining LMW-HA includes treating native HA under mildly acidic conditions to generate molecules containing a mixture of N-acetyl glucosaminyl and glucuronyl residues at their nonreducing ends. The terminal nonreducing glucosaminyl groups on the acid depolymerized LMW-HA can be removed with an exo-β-N-acetyl glucosamimidase to provide LMW-HA with glucuronyl residues at the nonreducing terminal of the molecules. See FIG. 2. The invention also provides LMW-HA molecules containing 4,5-unsaturated glucuronyl residues at the nonreducing terminal. See FIG. 3. One method for obtaining 4,5-unsaturated glucuronyl terminated HA fragments is by treatment of native HA with hyaluronidase.

The LMW-HA for use with the present invention consists of fragments wherein at least about 90% of the LMW-HA fragments have glucuronic acid or unsaturated glucuronic acid at their nonreducing terminal. Preferably, at least about 95% of the LMW-HA fragments for use with the invention have a glucuronic acid or an unsaturated glucuronic acid at their nonreducing terminal.

When using sonication, native HA can be dissolved in a suitable solvent, such as phosphate buffered saline, and the solution sonicated until the desired amount of depolymerization is obtained. See, for example, Kubo et al. Glycoconj. J, 1993, 10:435. The LMW-HA obtained by such treatment preferably has a molecular weight of about 10-20 Kd and contains mainly glucuronic residues at their nonreducing terminal end, i.e. greater than 95%.

In addition, LMW-HA fragments containing a mixture of N-acetylglucosaminyl and glucuronyl residues at their nonreducing ends can be obtained by treatment of HA under mildly acidic conditions. About 50% of the LMW-HA fragments obtained by this method have glucuronic acid at their nonreducing terminal. Subsequent to the acid treatment, the terminal nonreducing glucosaminyl groups can be selectively removed from the fragments with an exo-β-N-acetyl glucosamimidase (available from Sigma) to expose glucuronyl residues at the terminal end of the molecules. By varying the reaction conditions, the percentage of fragments with terminal nonreducing glucosaminyl groups can be controlled. For example, the reaction can be stopped by disrupting the enzyme with heat or pH at various points of completion to obtain the desired percentage of fragments with glucuronic acid at their nonreducing terminal.

In addition, LMW-HA with either N-acetyl-β-D-glucosamine or β-D-glucuronic acid at the reducing end can be chemically synthesized by methods known in the art. See Blatter G, Carbohydr. Res. 1996, 288:109-125 and Halkes K. M. Carbohydr. Res. 1998, 309: 161-164. For example, a suitably protected glucosamine-glucuronic acid disaccharide can first be prepared from the corresponding monosaccharides. The monosaccharides can be coupled by methods know in the art, such as by the use of an α-trichloroacetamidoglucopyranose as the glycosyl donor. The resulting disaccharide can then be repeatedly coupled with itself to form LMW-HA of varying size. For example, the anomeric protecting group on the glucuronic acid portion of the disaccharide can be selectively removed. A preferred protecting group for this position is the 4-methoxyphenyl group. For the reducing-end disaccharide, i.e. the first glycosyl acceptor, the anomeric position can be converted to a methoxy moiety, for example, by first converting the 4-methoxyphenyl group to anomeric hydroxyl by treatment with ceric ammonium nitrate. The resulting anomeric hydroxyl group can be converted to an α-trichloroacetimidate moiety by treatment with trichloroacetonitrile and DBU. The a-trichloroacetimidate moiety can be converted to the methoxy moiety by treatment with anhydrous methanol followed by treatment with trimethylsilyl triflate and triethylamine. For disaccharides to be used as glycosyl donors, the anomeric position can be converted to the α-trichloroacetimidate moiety as described above. The methoxy-protected disaccharide described above can be used as a glycosyl acceptor after selectively removing the protecting group at the 3 position of the glucosamine residue. A preferred protecting group for this position is the chloroacetyl group which can be removed by treatment with thiourea and pyridine in ethanol. The disaccharide donor can be coupled to the acceptor in an interative manner to produce LMW-HA, i.e., each successive coupling produces LMW-HA with an additional repeat unit. Additional preferred protecting groups for the glucosamine residue are a 4,6-O-benzylidene group and a 2-N-trichloroacetamido group. Additional preferred protecting groups for the glucuronic acid residue are 6- and 4-O-benzoyl groups and a C6 methyl ester. The use of these and alternative protecting groups is described in “Protective Groups In Organic Synthesis,” 2^{nd} Ed., by T. W. Greene and P. G. M. Wuts, 1991, John Wiley & Sons, Inc., which is incorporated herein by reference.

HA fragments can also be synthesized enzymatically using uridine diphosphate-sugars and HA synthetase. See, for example, De Luca et al. J. Am. Chem. Soc. 1995 117:5869-5870. These last two methods allow for the synthesis of LMW-HA having any percentage of glucuronic acid at its terminal. Thus, the formation of LMW-HA by enzymatic degradation, enzymatic synthesis or by chemical synthesis allows for the production of greater than about 98%, or greater than about 99% of the LMW-HA fragments having a glucuronic acid or an unsaturated glucuronic acid at their nonreducing terminal.

The LMW-HA may be coupled to a carrier by methods known in the art. See, for example, Dick and Beurret in Conjugate Vaccines, Cruse et al. eds., Contrib. Microbiol. Immunol., Basel, Karger, 1989, vol. 10, pp 48-114 and Jennings and Sood in Neoglycoconjugates: Preparation and Applications, Lee et al. eds., Chapter 10, pp 325-371, 1994, Academic Press, San Diego. The methods include reductive amination, coupling through the carboxylate moiety, the use of linkers, and the use of cyanogen bromide or its derivatives. When conjugating LMW-HA to a carrier, it is preferable to avoid alteration of the epitope at the nonreducing end of the polysaccharide. A preferred method of conjugating LMW-HA to carrier is by direct conjugation such as by reductive amination at the reducing terminal saccharide. For example, a reducing terminal end group may be selectively introduced into LMW-HA by reduction of the reducing terminal residue with, for example a borohydride, followed by periodate oxidation and reductive amination. See, for example, Jennings U.S. Pat. No. 4,356,170.

Methods which conjugate LMW-HA to a carrier at various locations along the back-bone of the LMW-HA can also be used. For example, LMW-HA can be activated by the use of periodate to generate aldehyde groups in the backbone residues of the polysaccharide and the activated LMW-HA is then treated with a carrier containing a free amino group in the presence of a reducing agent such as a borohydride. These methods also permit conjugation of more than one carrier molecule to a single LMW-HA which permits cross linking.

The polypeptide component of the conjugate molecules of the invention may be any physiologically tolerated protein or polypeptide which evokes a T cell dependent response when coupled to LMW-HA. The term polypeptide is intended to be a generic term that includes peptides, polypeptides and proteins including native, modified, or recombinant proteins. Examples of polypeptides useful as carriers include, but are not limited to, bacterial toxins, toxoids, porins, outer membrane proteins, and cross-reactive protein materials. Such polypetides include, but are not limited to, tetanus toxoid, diphtheria toxoid, pertussis toxoid, an immunogenic polypeptide derived from streptococci, an immunogenic polypeptide derived from influenza, an immunogenic polypeptide derived from meningococci, an immunogenic polypeptide derived from pneumococci, and an immunogenic polypeptide derived from E. coli In particular, tetanus toxoid, diphtheria toxoid, CRM₁₉₇, and porin polypeptides from, hemophilus, E. coli and neisseria, such as rPorB, are preferred. See for example U.S. Pat. No. 5,439,808.

The conjugate molecules prepared according to the present invention typically comprise a carrier polypeptide or protein to which is bound at least one low molecular weight hyaluronic acid fragment through a single binding site at the terminal end of the backbone of the polysaccharide fragment. Thus, the present invention provides the ability, if desired, to produce low molecular weight hyaluronic conjugate molecules wherein the polysaccharide component, except for one end, is unobscured by the carrier. These types of conjugates may be referred to as neoglycoproteins. See, for example, Dick and Beurret, supra. Alternatively, cross-linked conjugates may be formed according to the present invention. These types of conjugates may be referred to as lattice conjugates. See, for example, Dick and Beurret, supra.

The molecular ratio of LMW-HA to polypeptide or protein in the conjugate molecules of the invention is preferably between about 1 to about 100 molecules LMW-HA per molecule polypeptide or protein. More preferably the ratio is between about 10 and about 20 molecules LMW-HA or epitopes per molecule of polypeptide or protein. Alternatively, the ratio of LMW-HA to polypeptide or protein can be determined by weight. For example, the conjugate molecules of the invention are between about 10% and about 500% weight LMW-HA to weight polypeptide or protein. Preferably, the conjugates of the present invention are about 30% to about 100% weight LMW-HA to weight polypeptide or protein. In one embodiment, very low molecular weight hyaluronic acid, i.e., less than about 20 repeat units, is used to form conjugates which increases the density of the epitope. Variations in LMW-HA/polypeptide or protein ratio may be achieved by adjusting the conjugation conditions, especially the ratio of the starting components in the conjugation reaction.

In addition to providing conjugate molecules comprising low molecular weight hyaluronic acid conjugated to polypeptide or protein, the present invention also encompasses multivalent conjugates and pharmaceutical compositions and vaccines comprising the multivalent conjugates wherein different polysaccharides are conjugated to a single polypeptide. For example, low molecular weight hyaluronic acid may be bound to polypeptide or protein in various combinations with other polysaccharides. Examples of such polysaccharides are the capsular polysaccharides from Haemophilous influenzae type b; group B streptococcus type Ia, and Ib, II, III, IV, V, VI, and VIII; meningococcal groups A, B and C; and group A streptococcus polysaccharide.

The immunogenic conjugates according to the present invention provide useful pharmaceutical compositions, such as vaccines, which are important for providing protection against infection by HA containing bacteria, such as group A and group C streptococci, in mammals, particularly humans and horses. Further, these vaccines are useful for administration to pregnant females as a means of providing protective antibodies to a neonate prior to birth.

The immunogenic compositions of the invention may be used as a means for raising monoclonal, polyclonal, or anti-idiotypic antibodies useful for prophylactic, therapeutic and diagnostic purposes. Diagnostics are particularly useful in monitoring and detecting various infections and diseases caused by HA containing bacteria such as group A or group C streptococci. The immunogenic compositions of the present invention may be used as an immunogen for use in both active and passive immunogenic protection in those individuals infected or at risk of infection especially by group A or group C streptococci. The bactericidal antibodies used for passive protection are produced by immunizing a mammal with any of the immunogenic compositions of the invention and then recovering bactericidal antibodies. The bactericidal antibodies for use with the present invention may be present in serum, a partially purified fraction such as a gamma globulin fraction, or purified antibodies. For example, IgG can be purified from crude protein mixtures, such as serum or ascitic fluid, by using protein A- or protein G-agarose. Protein A binds to the Fc portion of IgG. Protein G also binds to the Fc region, but can also bind to the Fab region, making it useful for purification of F(ab)′₂ fragments of IgGs. Crude samples of IgGs can be purified using a protein A— or protein G-agarose columns. Serum samples, ascitic fluid or tissue culture supernatant should be diluted at least 1:1 with buffer before applying to a column. After applying the sample, the column is washed with a wash buffer, e.g. 20 mM sodium phosphate, 150 mM NaCl, pH 7.4, until most of the impurities are removed. The IgG is then eluted with elution buffer, e.g. 100 mM glycine, pH 3.0. The IgG can then be concentrated by diafiltration or further purified by ionic exchange or size exclusion chromatography.

“Humanized” antibodies (including chimeric and CDR-grafted antibodies), antibody fragments, and especially bi-specific antibodies based on the claimed monoclonal antibodies are within the contemplation of the present invention, as are recombinant antibody-related products produced in procaryotic or eucaryotic cells. For example, antibody fragments, such as Fab and F(ab′)₂ fragments, can be produced in culture by host cells such as E. coli, yeast, insect and mammalian cells upon determination of structural (sequence) information for the variable regions of the antibodies of the invention. See, for example, U.S. Pat. No. 6,180,377. Sequence information for the variable regions also enables preparation of CDR-grafted antibodies. Moreover, chimeric antibodies (e.g., mouse/human antibodies) may be prepared using transformed mouse myeloma cells or hybridoma cells and bi-specific antibodies may be produced by hybrid hybridoma cells.

The pharmaceutical compositions and vaccines of the invention are typically formed by dispersing the low molecular weight hyaluronic acid and/or conjugate in a suitable pharmaceutically acceptable carrier, such as physiological saline, phosphate buffered saline or other injectable liquids. The pharmaceutical composition or vaccine may be administered parenterally, for example subcutaneously, intraperitoneally, or intramuscularly. Additives customary in pharmaceutical compositions such as vaccines may also be added; for example, stabilizers, such as lactose or sorbitol, and adjuvants such as aluminum phosphate, aluminum hydroxide, aluminum sulphate, monophosphoryl lipid A, QS21, or stearyl tyrosine. Such pharmaceutical compositions may comprise the low molecular weight hyaluronic acid, its conjugate, or antibodies to low molecular weight hyaluronic acid and/or to its conjugate. The compositions may be administered alone or in combination with at least one other agent, such as an adjuvant or a stabilizing compound, which may be administered in any sterile, biocompatible pharmaceutical carrier, including, but not limited to saline, buffered saline, dextrose, and water. The compositions may be administered alone to a patient, or in combination with other agents, drugs, hormones, or biological response modifiers.

The pharmaceutical compositions and vaccines are administered in amounts sufficient to provoke an immunogenic response. Dosages will normally be within the range of about 0.1 to 50 μg of conjugate molecule per kilogram of body weight. Dosages may be adjusted based on the size, weight, or age of the individual and is well within the level of skill in the art. A series of doses may be given for optimum immunity. The antibody response in an individual can be monitored by determining antibody titer or bactericidal activity and the individual may be boosted, if necessary, to enhance the response.

The present invention provides compositions comprising antibodies, such as purified antibodies, gamma globulin fractions and serum useful for providing passive immunity to mammals infected or in danger of being exposed to HA containing bacteria, especially group A or group C streptococci. Among the many pathologies caused by streptococci is the invasive soft tissue infection which is associated with significant morbidity and mortality. See, for example, Ashbaugh et al. J. Clin. Invest., 1998, 102:550. The IgGs, antibody fragments, antibodies, gamma globulin fractions, and serum provided by the present invention are useful for inhibiting or preventing infection from HA containing bacteria, such as group A and group C streptococci and the resulting tissue necrosis and other pathologies resulting from such infection. Pharmaceutical compositions comprising antibodies, such as purified antibodies, gamma globulin fractions and serum are typically formed by dispersing the antibodies in a suitable pharmaceutically acceptable carrier, such as physiological saline, phosphate buffered saline or other injectable liquids. The pharmaceutical compositions may be administered parenterally, for example subcutaneously, intraperitoneally, or intramuscularly. Additives customary in pharmaceutical compositions may also be added. The compositions may be administered alone or in combination with at least one other agent, such as a stabilizing compound, which may be administered in any sterile, biocompatible pharmaceutical carrier, including, but not limited to saline, buffered saline, dextrose, and water. The compositions may be administered alone to a patient, or in combination with other agents, drugs, hormones, or biological response modifiers.

The pharmaceutical compositions comprising antibodies are administered in amounts sufficient to inhibit bacterial infection. Dosages may be adjusted based on the size, weight, or age of the individual and is well within the level of skill in the art. A series of doses may be given for optimum immunity. The dose response in an individual can be monitored by determining antibody titer or bactericidal activity and the individual may be given additional doses, if necessary, to enhance the response.

Antibodies prepared according to the present invention are also useful for preparing various immunoreagents and for use in immunoassays. For example, for immunoassays the low molecular weight hyaluronic acid fragments or their conjugates may be immobilized either directly or through a linker, such as a polypeptide linker, to a solid support. Methods similar to those used to make conjugates can be used for immobilization. The antibodies can then be used in various immunoassay systems known to those in the art including, radioimmuno assays and ELISA for detecting the presence of HA containing bacteria, such as group A or group C streptococci. Such assays may be used for diagnosing the presence of infection in individuals by assaying for the presence of group A or group C streptococcal antibodies in serum or other body fluids.

The examples presented herein are meant to exemplify the various aspects of carrying out the invention and are not intended to limit the scope of the invention in any way. All publications, patents and articles referred to within this specification are hereby incorporated in their entirety into this specification.

### EXAMPLE 1

### Preparation of LMW-HA

Depolymerization of Hyaluronic Acid by Acid Hydrolysis

Hyaluronic acid (100 mg, Lifecore lot 1-9062-5) was added to a 10 ml solution of 0.05 N HCl. The mixture was heated at 80° C. for 2 hours, and stirred in order to dissolve the entire solid. The sample was then heated for another 1.5 hours at 100° C. The depolymerization was monitored by removal of aliquots from the reaction mixture at various times and analysed on a Bio-Rad system (Biologic) equipped with a Superose® 12 HR 10/30 column (Pharmacia). The solution was neutralized with 0.5 N NaOH, then dialysed with a Diaflo® membrane of molecular weight cut-off (MWCO) 3,500 and lyophilized. The product was molecular size fractionated through a Superdex® 200 PG (Pharmacia) column to yield 65 mg of solid product. ¹H-NMR analysis of the samples at 500 MHz confirmed the structure of the disaccharide-repeating unit of hyaluronic acid (HA). The average molecular weight of the generated fragment was estimated by size-exclusion chromatography coupled with multiangle laser light scattering photometry (SEC MALLS) to be about 12,000 daltons.

Preparation of HA Oligosaccharides Containing D-Glucuronic Acid at Their Nonreducing Ends

Hyaluronic acid (100 mg, Lifecore lot 1-9062-5) is treated with 0.1 N HCl at 80° C. for 10 hours. The solution is neutralized with 0.5 N NaOH and desalted through a Sephadex® G-20 (Pharmacia) column eluted with water. The desalted product is freeze dried and treated with β-N-acetyl glucosamimidase (EC 3.2.1.30; Calbiochem) to generate LMW-HA fragments with about 4 to about 20 repeat units and containing D-glucuronic acid at their nonreducing ends. The structure of the oligosaccharides is confirmed by NMR spectroscopy and methylation analysis.

Depolymerization of Hyaluronic Acid by Sonication

Hyaluronic acid (100 mg, Lifecore lot 1-9062-5) was dissolved in 20 ml of 10 mM PBS buffer, and the suspension stirred until dissolved. The sample was sonicated with a Branson sonicator model 450, (sonication settings: Output control: 3; Duty cycle: 50%; temperature: 2° C.) for 18 hours. After dialysis and lyophilization, 57 mg of solid product was recovered. The average molecular weight of the resulting sonicated hyaluronic acid was determined to be 18,000 daltons by SEC-MALLS using a MiniDawn instrument (Wyatt technology, Santa Barbara, Calif.) and a Superose® 12 HR 10/30 column (Pharmacia). ¹H-NMR analysis of the samples at 500 MHz confirmed the structure of the disaccharide-repeating unit of hyaluronic acid.

### EXAMPLE 2

### Preparation of LMW-HA Conjugates

Reduction of Acid-Treated Hyaluronic Acid with NaBH4

Hyaluronic acid, depolymerized with hydrochloric acid (65 mg), was dissolved in 6.5 ml of deionized water. The pH was adjusted to 10 with 0.5 N NaOH and 64.5 mg of NaBH₄ added to the solution. The reaction mixture was held at room temperature for 2 hours. The excess NaBH₄ was destroyed with 1 M acetic acid. Dialysis against deionized water with a Diaflo® membrane of MWCO 3,500 followed by lyophilization yielded 35 mg of solid product.

Periodate Oxidation of Reduced Acid-Treated Hyaluronic Acid

Two 18 mg samples of the reduced and sized polysaccharides were dissolved in 1.3 ml and 0.87 ml of a 10 mM aqueous solution of NaIO₄ to achieve a degree of oxidation (d.o.) of 10 and 20 percent respectively. The reactions were stirred in the dark for 2 hours at room temperature, and each was quenched with 20 μl of ethylene glycol. The reaction mixtures were then dialyzed and lyophilized to provide 17 mg of solid product.

Preparation of Acid-Treated Hyaluronic Acid-Tetanus Toxoid Conjugate (LMW-HA/TT)

Periodate-oxidized (d.o. 10% and 20%) acid-treated hyaluronic acid (10 mg of each respectively) and purified tetanus toxoid monomer (5 mg for each sample, Statens Serum Institute, Copenhagen, Denmark) were dissolved in 0.5 mL of 0.2 M sodium phosphate, pH 7.4. Recrystallized sodium cyanoborohydride (10 mg for each sample) was added and the mixture held at room temperature overnight. The progress of the reaction was monitored at various times using a Bio-Rad (Biologic) system equipped with a Superose® 12 HR 10/30 column (Pharmacia). Conjugation of polysaccharide to protein was indicated by a progressive increase of a UV (280 nm) peak eluting in the void volume of the column. After conjugation was completed, 10 mg of NABH₄ in 1 ml of 0.1 N NaOH was added to each sample in order to reduce any remaining unconjugated aldehyde. The conjugate was purified by passage over a column (1.6×60 cm) of Superdex® 200 PG (Pharmacia) eluting with 10 mM PBS containing 0.01 percent thimerosal. Fractions corresponding to the void-volume peak were pooled and stored at 4° C. They were designated conjugates 1 and 2 for 10 and 20 percent oxidation in their polysaccharides, respectively.

Periodate Oxidation of Sonicated Hyaluronic Acid

Two samples of 26 mg and 30 mg of the sonicated polysaccharides were each dissolved in 2 and 1.5 ml of deionized water, respectively and treated with 0.65 ml and 1.5 ml of 10 mM aqueous NaIO₄ to reach degrees of oxidation (d.o.) of 10 and 20 percent, respectively. The reactions were stirred in the dark for 2 hours at room temperature, and each reaction was quenched with 20 μl of ethylene glycol. The solutions were then dialyzed and lyophilized to provide 24 and 25 mg of product, respectively.

Preparation of Sonicated Hyaluronic Acid-Tetanus Toxoid Conjugate (LMW-HA/TT)

Sonicated and periodate-oxidized HA (7 mg of each sample, d.o. 10% and 20%) and purified tetanus toxoid monomer (3.5 mg for each sample) were dissolved in 350 μl of 0.2 M sodium phosphate at pH 7.4. Sodium cyanoborohydride (7 mg for each sample) was added, and the mixtures held at room temperature overnight. The progress of each conjugation reaction was monitored by removal of aliquots from the reaction mixture at various times and subsequent analysis on a Bio-Rad (Biologic) system equipped with a Superose® 12 HR 10/30 column (Pharmacia). Conjugation of polysaccharide to polypeptide was indicated by the progressive increase of a UV absorbing peak (280 nm) eluting in the void volume of the column. After conjugation was completed, NaBH₄ (10 mg in 1 ml of 0.1 N NaOH for each sample) was added to the reaction mixtures to reduce any remaining unconjugated aldehyde. The conjugates were purified by passage over a Superdex® 200 PG (Pharmacia) column, eluted with 10 mM PBS containing 0.01 percent thimerosal. Fractions corresponding to the void-volume peak were pooled and stored at 4° C. and were designated conjugates 3 and 4 for 10 and 20 percent oxidation in their polysaccharides, respectively.

Coupling of Sonicated Hyaluronic Acid to Recombinant Class 3 Neisserial Porin (LMW-HA/rPorB)

Sonicated and periodate-oxidized hyaluronic acid (20 mg, 20% d.o.) and rPorB (10 mg) were dissolved in 717 μl of 0.25 M HEPES buffer, pH 8.5, containing 0.25 M NaCl and 0.05 percent Zwittergent Z 3,14 (Calbiochem, San Diego, Calif.). Sodium cyanoborohydride (20 mg) was added, and the mixture incubated at 37° C. for 1 day. After the conjugation was completed, 10 mg of sodium borohydride in 1 ml of 0.1 N NaOH was added to the reaction mixture to remove any remaining aldehyde. The conjugate was purified by passage over a column of Superdex® 200 PG (Pharmacia), eluted with 10 mM PBS containing 0.01 percent thimerosal. Fractions corresponding to the void-volume peak, as monitored by UV absorbance at 280 nm, were pooled and stored at 4° C. and labeled as conjugate 5.

Preparation of Hyaluronic Acid-Human Serum Albumin Conjugates (LMW-HA/HAS) as ELISA Coat Antigens

Both acid-treated and sonicated periodate-oxidized hyaluronic acids with a d.o. of 10 percent, and Human Serum Albumin (HSA, Fluka) were dissolved in 0.5 ml of sodium phosphate buffer, pH 7.4. Sodium cyanoborohydride was added, and the mixtures incubated at 37° C. for 1 day. After conjugation was completed, sodium borohydride in 0.1 N NaOH was added to the reaction mixtures as described for the other conjugates to remove any remaining aldehyde. Conjugates were dialyzed and lyophilized.

TABLE 1
Physio-Chemical Characteristics of LMW-HA/Protein Conjugates:

| | | Mode of | | % HA in |
|---|---|---|---|---|
| Conjugates | Carrier | Fragment | PS d.o. | Conjugate |
| 1 | TT | Sonication | 10 | 30 |
| 2 | TT | Sonication | 20 | 28 |
| 3 | TT | Acid hydrolysis | 10 | 31 |
| 4 | TT | Acid hydrolysis | 20 | 20 |
| 5 | rPorB | Sonication | 20 | 16 |

The hyaluronic acid and protein contents in the conjugates were measured by the carbazole (for uronic acids) (Bitter, T. 1962 Anal. Biochem. 4: 330) and coomassie (BioRad) assays respectively.

### EXAMPLE 3

### Isolation of the Hyaluronic-Acid Oligosaccharide Inhibitors

Hyaluronate lyase (EC 4.2.2.1) from Streptomyces hyalurolyticus (Sigma Biochemicals), the content of 3 ampoules in 10 mM PBS, was added to sonicated hyaluronic acid (60 mg) and incubated at 37° C. for 1.5 hours. The reaction was stopped by boiling the reaction mixture at 100° C. for one minute in a water bath. The progress of the enzymatic digestion was monitored by removal of aliquots of the reaction mixture and analysis on a BIO-RAD system (Biologic) equipped with a Superdex® peptide column (Pharmacia), with 10 mM PBS as eluant at a flow rate of 0.75 ml/min. The solution was stored at 4° C. until further purification.

Isolation of the oligosaccharides was performed by anion-exchange chromatography with a Mono-Q HR 5/5 column (Pharmacia) using a HPLC 1090 (Hewlett Packard 1090 Series II) system equipped with a diode-array detector, a programmable auto-injector, a fraction collector, and the Hewlett Packard Chemstation software program for system control and data acquisition/processing. A step-gradient of sodium chloride in Tris-HCL buffer was used for the separation. Two oligosaccharide fractions corresponding to a dimer (DP2) and a tetramer (DP4) eluting, respectively, between 18 to 26 minutes and between 28 to 31 minutes were collected, lyophilized and desalted using a Sephadex G-10 column (Pharmacia) and deionized water as eluant. The structure of the oligosaccharides was confirmed by examination of their ¹H-NMR spectra at 500 MHz. The DP2 oligosaccharide corresponded to Δ4,5-β-GlcU-(1,3)-D-GlcNAc, and the DP4 to Δ4,5-13-GlcU-(1,3)-β-D-GlcNAc-(1,4)-β-D-GlcU-(1,3)-β-D-GlcNAc.

### EXAMPLE 4

### Serological Studies: Immunospecificity of Rabbit Antisera to Low Molecular Weight Hyaluronic Acids (LMW-HAs)

Immunogenicity Studies

New Zealand white rabbits were immunized subcutaneously, 3 times at 21 day intervals (days 0, 21, and 41) with 10 μg conjugated polysaccharide per dose of LMW-HA/TT in Freund's complete adjuvant for the first dose and incomplete Freund's adjuvant for the second and third doses. Rabbits were ear bled at days 21, 31 and 41 and a cardiac puncture test was performed 10 days after the third immunization.

Titration of Rabbit Antisera on LMW-HA/HSA Coated Plates

Microtiter plates (NUNC Polysorp) were passively coated with either sonicated LMW-HA/HSA or acid hydrolyzed LMW-HA/HSA conjugate (approximately 25 ng PS in 100 μL/well) diluted in PBS (50 mM sodium phosphate, 150 mM NaCl, pH=7.4) for one hour at 37° C. After washing the plates with PBS+0.05% Tween® 20 (PBS Tween, pH=7.4), the plates were post-coated with 150 μL/well PBS+0.1% Bovine Serum Albumin (PBS+BSA, pH 7.4) for one hour at room temperature. After the post-coat, the plates were again washed and stored at 2-8° C. until used.

Rabbit antisera were diluted serially in PBS Tween in wells of microtiter plates coated with either sonicated LMW-HA/HSA or acid hydrolyzed LMW-HA/HSA to a final volume of 100 μL/well and incubated for one hour at room temperature. The plates were washed with PBS Tween and 100 μL of a goat anti-rabbit IgG-Horseradish Peroxidase conjugate (Kirkegaard and Perry Laboratories) diluted 1:2,500 in PBS Tween was added to each well. Following a one-hour incubation at room temperature, the plates were again washed and 100 μL of TMB Substrate Solution (KPL) was added to each well. The plates were incubated for five to ten minutes at room temperature and color development was stopped by the addition of 100 μL of One Component Stop Solution (KPL) to each well. The optical density of each well was read at 450 nm, and titration curves were generated for each condition.

Inhibition of Binding of Rabbit Antisera to Sonicated LMW-HA/TT on LMW-HA/HSA Coated Plates

Microtiter plates were coated as above. Rabbit anti-sonicated LMW-HA/TT antisera were titered on plates coated with sonicated LMW-HA/HSA conjugate. The dilution corresponding to approximately one-half of the maximum signal was chosen as appropriate for the inhibition studies. The rabbit antisera were diluted into PBS Tween. Inhibitors were serially diluted in buffer containing the dilute antisera in Titertubes® (Bio-Rad) and 100 μL of each sample were taken from the Titertubes® and added directly to wells of coated microtiter plates. Samples were incubated in the microtiter plates for one hour at room temperature. The microtiter plates were washed with PBS Tween, then 100 μL of goat anti-rabbit IgG-HRP conjugate (KPL) diluted 1:2,500 in PBS Tween were added to each well. The plates were incubated for one hour at room temperature, and washed with PBS Tween. 100 μL of TMB Substrate Solution (KPL) was added to each well. The plates were incubated at room temperature for five to ten minutes and color development was stopped with the addition of 100 μL to each well of One Component Stop Solution (KPL), and the absorbance at 450 nm was read. Inhibition was determined as percent of maximum signal achieved with dilute antiserum in the absence of any inhibitor.

Immune Response of New Zealand White Rabbits

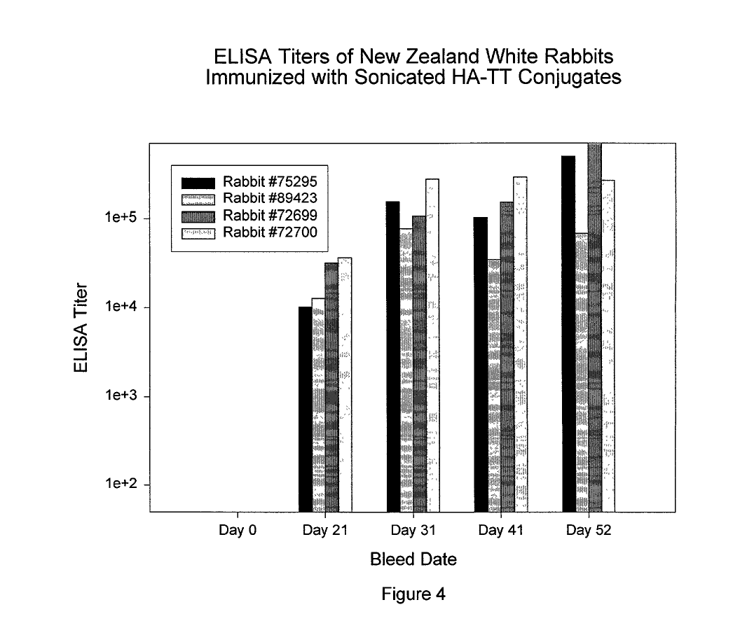

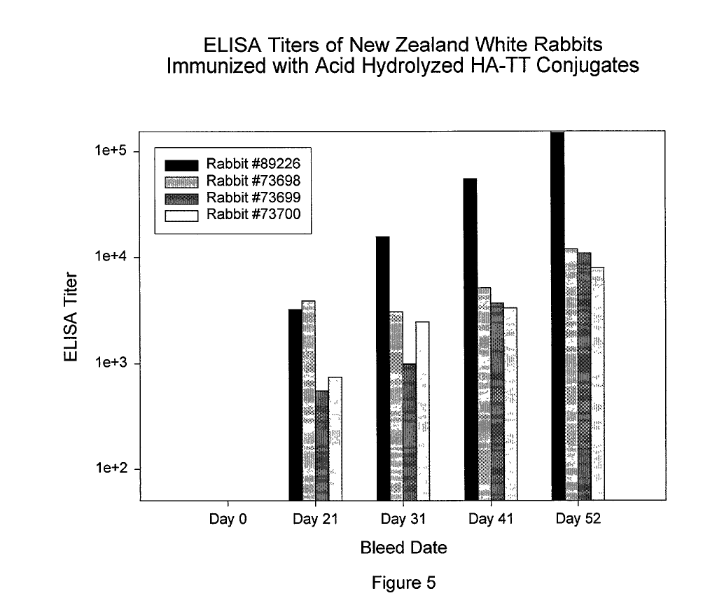

Eight New Zealand White rabbits were immunized with three subcutaneous injections of either sonicated Hyaluronic Acid-Tetanus Toxoid (LMW-HA/TT) or acid hydrolyzed Hyaluronic Acid-Tetanus Toxoid (LMW-HA/TT) conjugates (four animals for each conjugate). FIG. 4 displays the immune response for each individual rabbit for the sonicated LMW-HA/TT conjugate immunogen. All four animals responded to the hyaluronic acid with ELISA titers in excess of 50,000. FIG. 5 shows the immune response for the individual rabbits immunized with acid hydrolyzed LMW-HA/TT conjugate. All four individual animals responded to the hyaluronic acid with ELISA titers of approximately 10,000, or greater.

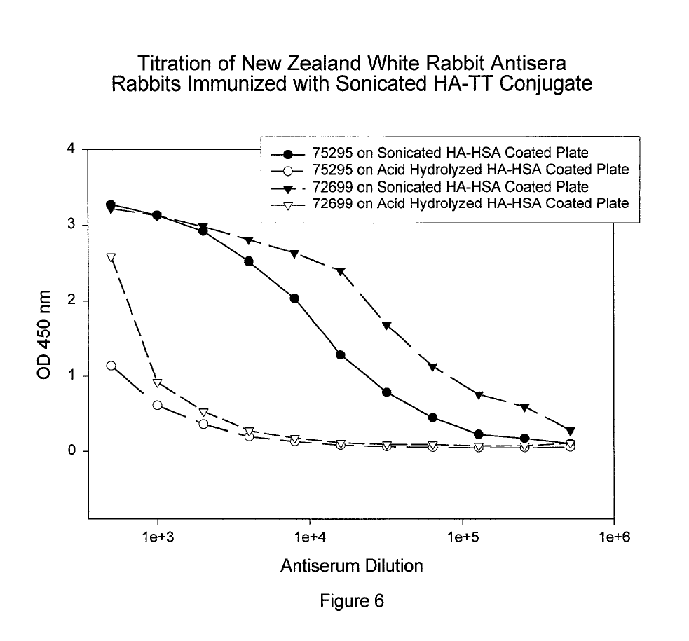

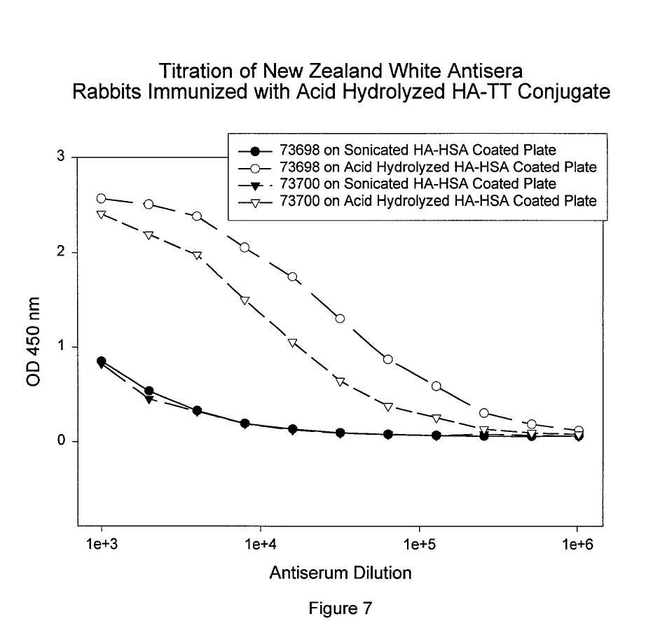

The immune response of the rabbits was demonstrated to be specific depending on the nature of the conjugate used for the immunizations. FIG. 6 shows that antibodies from animals that were immunized with the sonicated LMW-HA/TT conjugate generally reacted more readily to plates coated with sonicated LMW-HA/HSA, rather than acid hydrolyzed LMW-HA/HSA. There is at least an order of magnitude difference in the immunoreactivity between these different coating antigens. The converse holds true for the animals immunized with the acid hydrolyzed LMW-HA/TT conjugate. FIG. 7 shows an order of magnitude preference for these antisera to the acid hydrolyzed LMW-HA/HSA conjugate as opposed to the sonicated LMW-HA/HSA solid phase.

Specificity of Antisera Generated by Sonicated LMW-HA/TT in New Zealand White Rabbits.

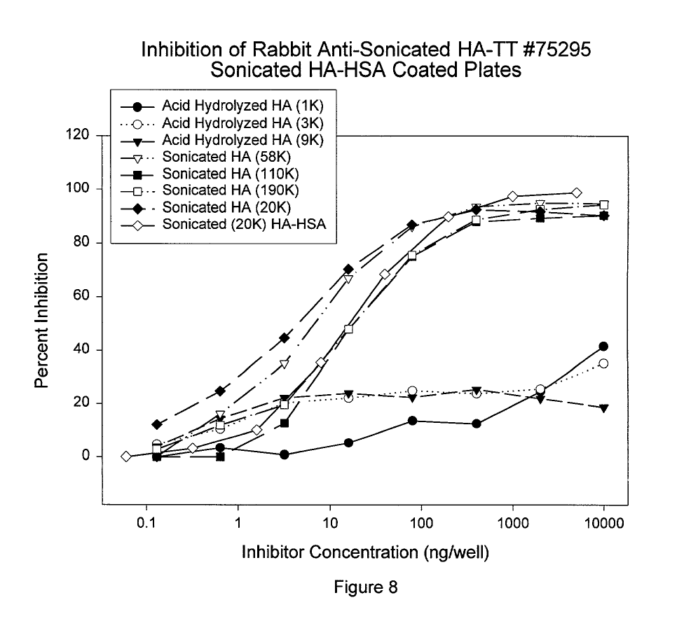

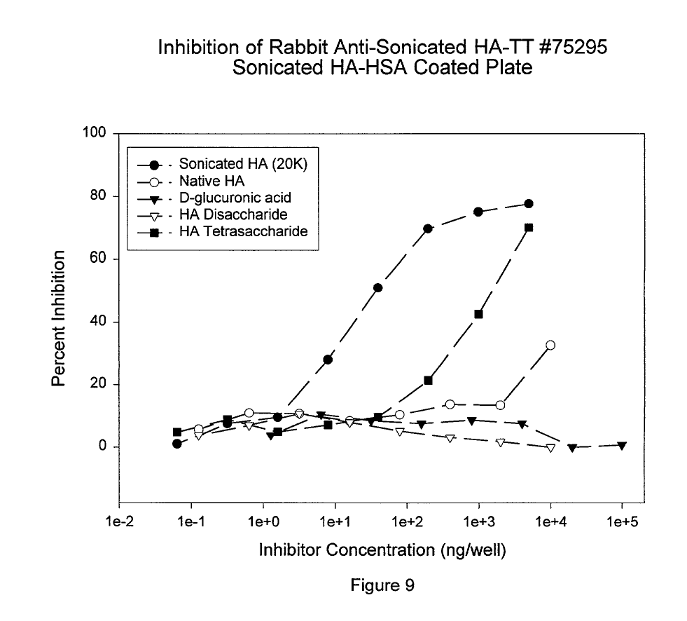

Specificity of antisera produced in rabbits by immunization with the sonicated LMW-HA/TT conjugate was examined by microtiter plate inhibition studies. Results of inhibition studies using antisera from rabbit #75295 immunized with sonicated LMW-HA/TT are represented in FIGS. 8 and 9. FIG. 8 indicates several species of sonicated HA as well as several species of acid hydrolyzed HA used as inhibitors. In this experiment, all forms of the acid hydrolyzed HA were poor inhibitors of antibody binding to the coated sonicated LMW-HA/HSA. Regarding the sonicated species of the inhibitors, the trend appears that the smaller the HA fragment produced by sonication, the more efficient that fragment is as an inhibitor. This indicates that the smaller HA molecules present more epitopes per unit mass than the larger species.

The results presented in FIG. 9 further elucidate this finding. Herein, the very -high molecular weight native HA is seen as a very poor inhibitor, almost three orders of magnitude less than the 20 kD HA used as the immunogen. Smaller species were also used as inhibitors in this experiment. The tetrasaccharide form of HA was a relatively efficient inhibitor, while the disaccharide and D-glucuronic acid forms did not inhibit the antibody binding to the solid phase at all.

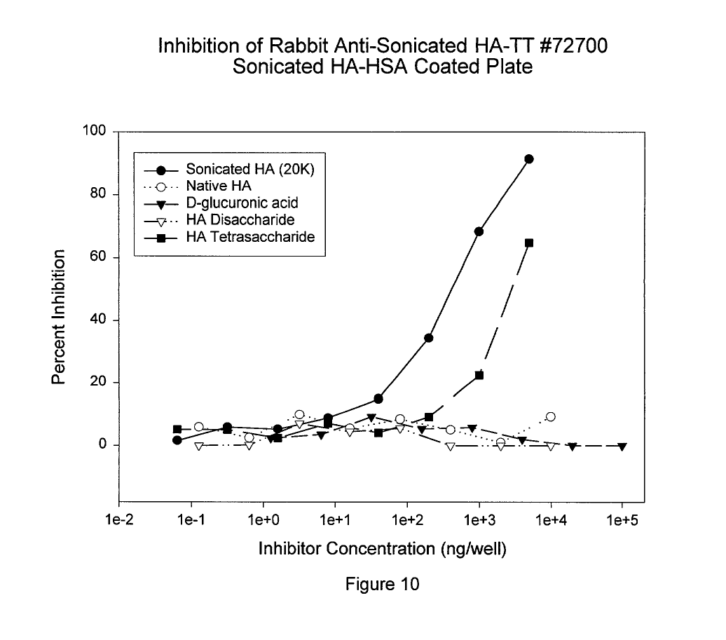

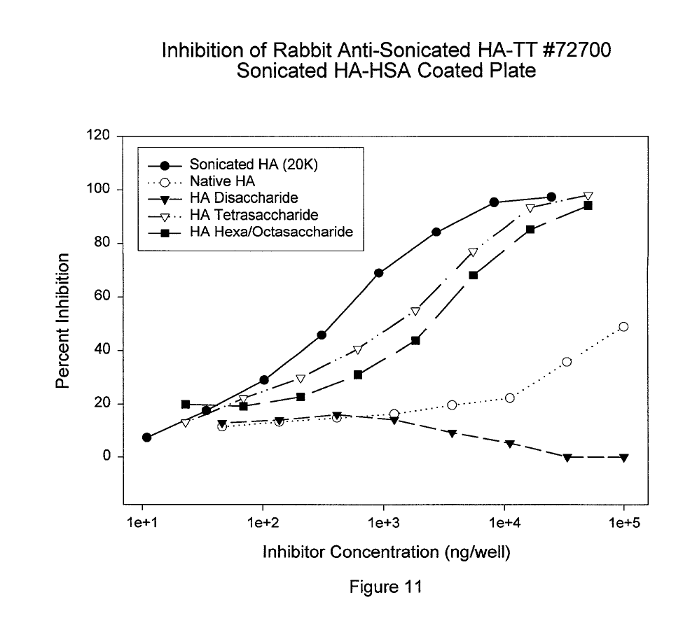

Results from inhibition studies performed using antiserum from rabbit #72700, which was also immunized with the sonicated LMW-HA/TT conjugate, are presented in FIGS. 10 and 11. These results are quite similar to those obtained from rabbit #75295 as previously described. FIG. 10 displays results similar to those seen in FIG. 9. That is, the tetrasaccharide form of the HA and the sonicated (20K) immunogen form of the HA inhibit well, while the disaccharide and native forms of HA inhibit poorly. The relationship between the size of the HA and immunoreactivity was further examined using a hexasaccharide form of HA. These results are shown in FIG. 11. The results indicate that this form of HA is capable of complete inhibition of antibody binding. From these studies it is apparent that at least the tetrasaccharide form (two repeating subunits) of HA is necessary for complete inhibition. The disaccharide form of HA is insufficient for inhibition and that the native form of the molecule is not an optimal inhibitor. Therefore, a reduction in the size of the large native HA molecule is preferable for immune recognition.

### EXAMPLE 5

### Immunogenicity Studies in Mice

Antisera Produced in Mice to Hyaluronic Acid-Protein Conjugates

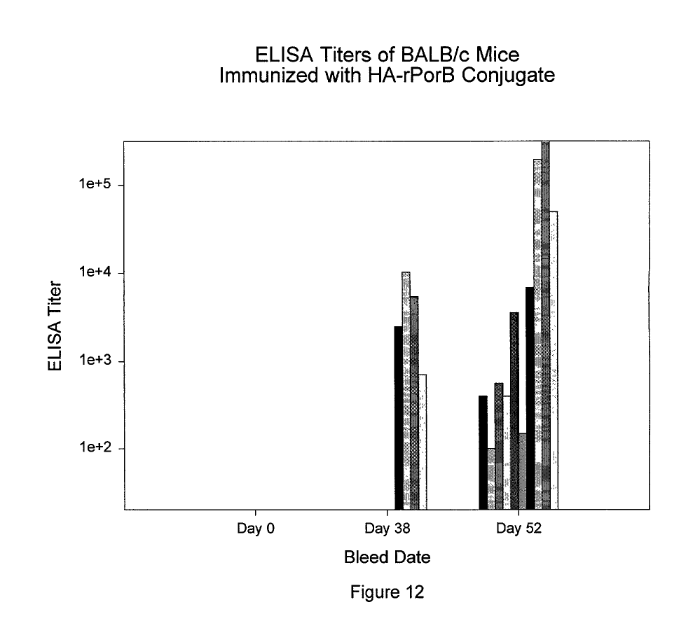

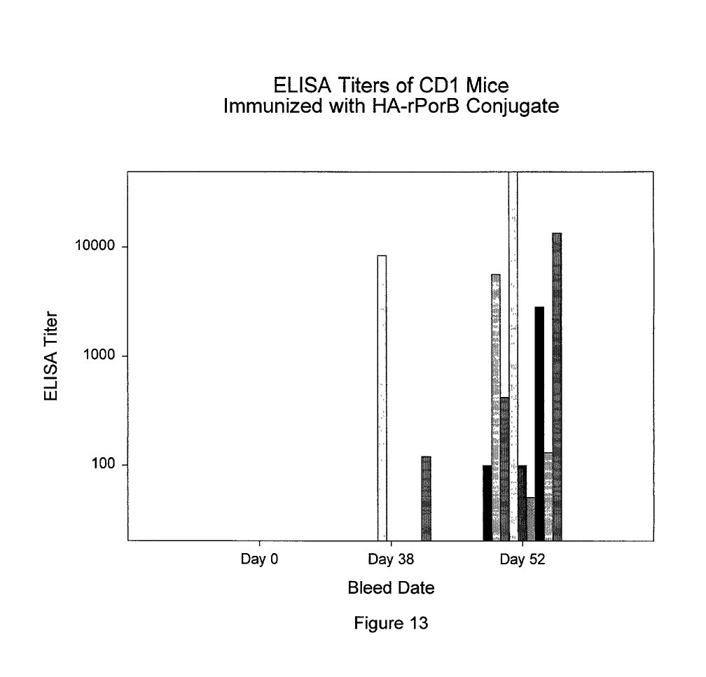

Both BALB/c and CD1 mice were immunized with three injections of LMW-HA/protein conjugate vaccines. The conjugates were either sonicated LMW-HA/TT, as with the rabbits, or sonicated HA conjugated to rPorB (LMW-HA/rPorB). The LMW-HA/TT conjugate vaccine produced no immune response greater than that of the negative control. However, the LMW-HA/rPorB conjugate produced measurable ELISA titers in 100% of the animals immunized. These data are shown in FIGS. 12 and 13 for BALB/c mice and CD1 mice, respectively.

### EXAMPLE 6

### Protection Experiments

Passive Immunization

Rabbit antisera to LMW-HA/TT conjugates were used for protection assay in passive immunization in adult Balb/c mice. The rabbit antisera (0.5 ml) diluted by half in sterile PBS was injected intraperitonealy (IP) two hours before challenge (IP) with a LD90 challenge dose of group A streptococcus (GAS) type 6 or type 3. The antisera of a rabbit immunized with phosphate buffer saline (PBS) and complete Freund's adjuvant (CFA) as well as a rabbit antiserum raised against the group A streptococcal carbohydrate conjugated to tetanus toxoid were included in the protection experiment as control sera. Survival was followed for 10 days after challenge.

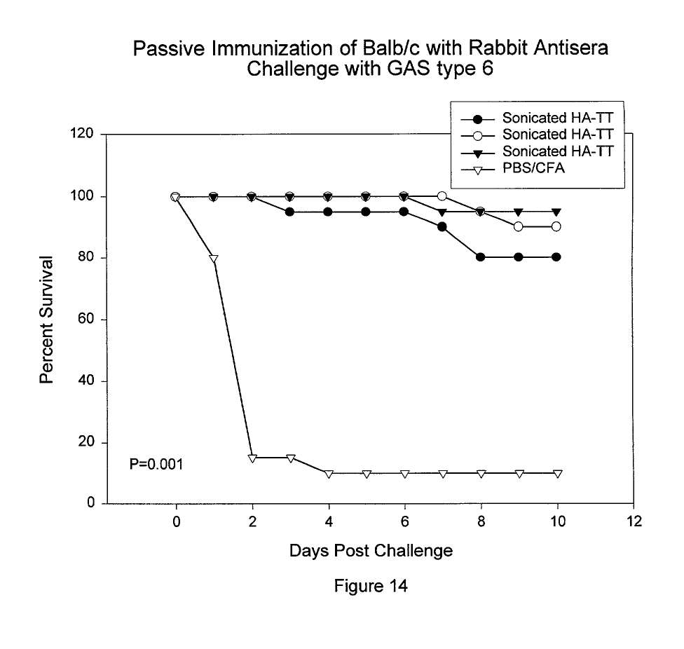

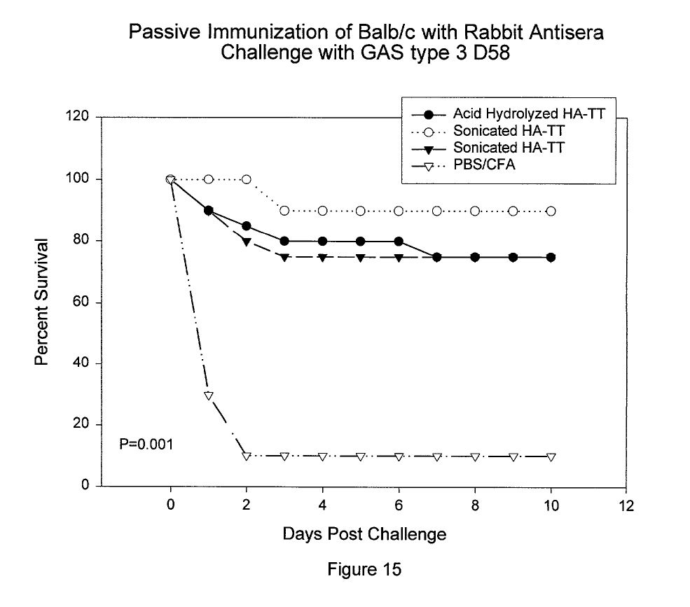

The LD90s were previously determined after immunization of Balb/c mice with rabbit antisera immunized with PBS/CFA followed 2 hours later by an IP challenge with a range of doses of GAS type 6 or type 3. The LD90 for GAS type 6 and type 3 was determined to be respectively 5×10³ and 2×10⁵ cfu/mL. The results of the challenge experiments are shown in FIGS. 14 and 15.

We claim:

1. An immunogenic conjugate molecule comprising hyaluronic acid covalently bound to an immunologically-suitable polypeptide carrier.

2. The immunogenic conjugate according to claim 1, wherein greater than about 50% of the hyaluronic acid molecules possess a nonreducing terminal glucuronic acid and/or unsaturated glucuronic acid residue.

3. The immunogenic conjugate according to claim 2, wherein the hyaluronic acid is a low molecular weight hyaluronic acid with a molecular weight of about 400 Kd or less and a molecular weight of about 600 daltons or more.

4. The immunogenic conjugate according to claim 3, wherein at least 90% or greater of the low molecular weight hyaluronic acid fragments possess a nonreducing terminal glucuronic acid and/or unsaturated glucuronic acid residue.

5. The immunogenic conjugate according to claim 3, wherein at least 95% or greater of the low molecular weight hyaluronic acid fragments possess a nonreducing terminal glucuronic acid and/or unsaturated glucuronic acid residue.

6. The immunogenic conjugate according to claim 3, wherein at least 98% or greater of the low molecular weight hyaluronic acid fragments possess a nonreducing terminal glucuronic acid and/or unsaturated glucuronic acid residue.

7. The immunogenic conjugate according to claim 3, wherein at least 99% or greater of the low molecular weight hyaluronic acid fragments possess a nonreducing terminal glucuronic acid and/or unsaturated glucuronic acid residue.

8. The immunogenic conjugate according to claim 3, wherein the low molecular weight hyaluronic acid is about at least about 4 glycosyl residues in size.

9. The immunogenic conjugate according to claim 3, wherein the low molecular weight hyaluronic acid possess about 2 to about 20 disaccharide subunits.

10. The immunogenic conjugate according to claim 9, wherein the low molecular weight hyaluronic acid is about 2 to about 10 disaccharide subunits.

11. The immunogenic conjugate according to claim 3, wherein the polypeptide carrier is selected from the group consisting of tetanus toxoid, diphtheria toxoid, pertussis toxoid, an immunogenic polypeptide derived from streptococci, an immunogenic polypeptide derived from influenza, an immunogenic polypeptide derived from meningococci, an immunogenic polypeptide derived from pneumococci, and an immunogenic polypeptide derived from E. coli.

12. The immunogenic conjugate according to claim 3, wherein the polypeptide carrier is a porin from neisseria.

13. The immunogenic conjugate according to claim 3, wherein the conjugate is directly linked.

14. The immunogenic conjugate according to claim 3, wherein the conjugate elicits antibodies that bind an epitope comprising glucuronic acid or unsaturated glucuronic acid as the nonreducing terminal sugar of a low molecular weight hyaluronic acid.

15. The immunogenic conjugate according to claim 3, wherein the conjugate elicits antibodies that bind capsular hyaluronic acid present in bacteria.

16. The immunogenic conjugate according to claim 15, wherein the bacteria is group A streptococci or group C streptococci.

17. A pharmaceutical composition comprising the conjugate according to claim 3 and a pharmaceutically acceptable carrier.

18. The pharmaceutical composition according to claim 17, further comprising a physiologically acceptable adjuvant.

19. A method of preparing a low molecular weight hyaluronic acid—polypeptide conjugate molecule comprising covalently linking low molecular weight hyaluronic acid fragments to an immunologically-suitable polypeptide, wherein about 50% or greater of the low molecular weight hyaluronic acid fragments have a glucuronic acid and/or an unsaturated glucuronic acid residue at the nonreducing terminal.

20. The method according to claim 19, wherein the method comprising covalently linking a low molecular weight hyaluronic acid to an immunologically-suitable polypeptide comprises reductive amination.

21. A purified antibody which binds to the immunogenic conjugate molecule according to claim 3.

22. The purified antibody according to claim 21, wherein the low molecular weight hyaluronic acid fragments are at least about 4 glycosyl residues in size.

23. The purified antibody according to claim 22, wherein the hyaluronic acid fragments are at least about 4 glycosyl residues and no more than about 40 kD in size.

24. A pharmaceutical composition effective for treating or inhibiting group A streptococcal or group C streptococcal infection comprising an antibody selected from the group consisting of an antibody elicited by the composition according to claim 17, an antibody according to 21, or an antibody elicited by low molecular weight hyaluronic acid conjugated to a liposome.

25. A method of eliciting an antibody response in a mammal, said method comprising the step of administering to the individual mammal an amount of a pharmaceutical composition according to claim 17 in an amount which is sufficient to elicit an antibody response.

26. The method according to claim 25, wherein the mammal is a human.

27. The method according to claim 25, wherein the pharmaceutical composition is administered intramuscularly, subcutaneously, intraperitoneally or intravenously.

28. The method according to claim 25, wherein the pharmaceutical composition is administered in an amount of about 0.1 to about 50 micrograms per kilogram body weight.

29. A vaccine that elicits effective levels of anti-low molecular weight hyaluronic acid antibodies in humans comprising the immunogenic conjugate according to claim 3.

30. A method of inhibiting streptococcal infection in a mammal, comprising administering to the mammal a pharmaceutical composition according to claim 3 in an amount sufficient to inhibit infection.

31. A method of inhibiting progression of infection in a mammal by bacteria containing HA, comprising administering to the mammal a composition comprising the pharmaceutical composition according to claim 24 in an amount sufficient to inhibit progression of the infection.

32. The method according to claim 31, wherein the bacteria are group A streptococci or group C streptococci.

33. A diagnostic immunoassay kit for detecting infection by streptococci comprising an antibody according to claim 21.
